(19) United States
(12) Patent Application Publication
(10) Pub. No.: US 2023/0166260 A1
(43) Pub. Date: Jun. 1, 2023

(54) Apparatus for holding down respective membrane elements in respective wells of a multiwell plate

(71) Applicant: EUROIMMUN Medizinische Labordiagnostika AG, Lübeck (DE)

(72) Inventors: Bianca Huth, Lübeck (DE); Patrick Auer, Lübeck (DE); Philip Rosenstock, Lübeck (DE)

(73) Assignee: EUROIMMUN Medizinische Labordiagnostika AG, Lübeck (DE)

(21) Appl. No.: 17/692,151

(22) Filed: Mar. 10, 2022

(30) Foreign Application Priority Data
Mar. 11, 2021 (EP) 21162090.1

(51) Int. Cl. B01L 3/00

(52) U.S. Cl. CPC B01L 3/50853; B01L 2300/069; B01L 2200/027; B01L 2300/046

(57) ABSTRACT

An apparatus is provided for holding down membrane elements in respective wells of a multiwell plate, which has, at its top side, respective openings of the respective wells. The apparatus has, at its upper region, a support structure having respective openings, and respective corresponding retention elements which extend downwards from the respective openings of the support structure, so that respective retention elements protrude into respective wells when the support structure has been placed onto the top side of the multiwell plate. A retention element has multiple webs which extend downwards from the support structure and the ends of which are connected to one another at a bottom side of the apparatus by means of a connection element. Furthermore, a retention element has multiple openings between the webs, which openings extend continuously from the support structure right up to the connection element and which openings furthermore extend continuously from the top side of the multiwell plate right up to the connection element when the support structure has been placed onto the top side of the multiwell plate.

### CROSS-REFERENCE TO RELATED APPLICATIONS

The present application claims priority pursuant to 35 U.S.C. § 119(a) to EP patent application 21162090.1, filed Mar. 11, 2021, which is incorporated by reference herein in its entirety.

### BACKGROUND

So-called multiwell plates, which have a plurality of wells, a respective well having an opening at the top side of the multiwell plate, are known from the prior art. The respective wells are each designed for accommodation of a respective liquid solution. Furthermore, it is known that, within such a well, in addition to the liquid solution, at least one membrane element can be placed into the well or the liquid solution.

Such a membrane element preferably includes dried blood constituents of a patient sample. For the purpose of elution or detachment of such dried blood constituents from the membrane element, the membrane element is introduced or placed into the solution for some time. Such a solution can preferably be an aqueous solution such as, in particular, a so-called buffer. Such elution or detachment of dried blood constituents from the membrane element is done for the purpose of transferring an analyte present in the dried blood constituents into the solution.

In particular, the membrane element is composed of an absorptive membrane material. Such a membrane element is, for example, punched out or detached from a so-called dried blood spot card beforehand, which then yields a so-called “dried blood spot” (DBS). The patient blood was previously applied to the relevant region of the membrane element on the dried blood spot card as capillary blood for example. This can, for example, be done by pricking of a fingertip using a lancet, so that the patient can then drip capillary blood from the fingertip onto the relevant region of the membrane element of the dried blood spot card.

In the course of processing for the purpose of elution or detachment of the dried blood constituents from the membrane element by means of the solution or in the course of a method therefor, the multiwell plate can, after a first standing time, be preferably covered and be positioned on a so-called rotating shaker, on which the multiwell plate is then subjected to a shaking movement for some time. This can preferably support the extraction or the elution/detachment of the dried blood constituents from the membrane element by means of movement of the membrane element in the solution. Subsequently, at least a partial volume of the liquid containing blood constituents dissolved therein can then be aspirated from a well by means of a pipetting needle or aspiration needle and be transferred into a separate vessel. In the vessel, an analyte possibly present can then be detected by means of further steps of an analysis method. When the partial volume of the liquid is aspirated from the well, what may possibly occur here is undesired clogging of the needle, and so an incorrect quantity of the partial volume of the liquid will be aspirated and a processing result of the analysis method will possibly be distorted.

For many qualitative determinations in the field of analytics and especially laboratory diagnostics, it is only important that the presence or absence of an analyte can be correctly established, but not the concentration in which it is present in the blood of a patient. For example, this is the case when the blood sample is being tested to determine whether the patient is suffering from a metabolic disease associated with a defective gene. The blood sample must then merely be tested to determine whether such a gene is present, but not how much of the genetic material comprising the gene is contained in the sample.

In the case of other analytes, the concentration in which the analyte occurs in the blood must be established because a diagnostic reference concentration range is known. If the concentration of the analyte lies within said range, this indicates that the patient is healthy. For such semi-quantitative or else purely quantitative determinations, it is therefore important that the quantity or concentration of the analyte is correctly determined.

For the measurement of concentrations of certain analytes in the blood of patients, venous blood can in principle be drawn from the patient, preferably followed by processing of the blood to give serum. Besides the fact that this invasive procedure is uncomfortable for the patient and, to a lesser extent, even entails health risks, such as the risk of nausea or unconsciousness, the drawing of blood can only be carried out by qualified personnel such as a physician or at least a nurse or experienced laboratory technician. To this end, the patient must visit a medical practice or a hospital.

By contrast, obtaining capillary blood is much simpler and gentler. After a sharp object has been pierced into the fingertip or the earlobe of the patient, there is extravasation of some drops of capillary blood, which are taken up using a pipette and added to an absorptive sample support. Once the blood has dried on the support, to completeness after a few hours at room temperature if necessary, the support can be transported without further processing, in a very simple manner and even by letter post. It is thus unnecessary to visit the medical practice. Owing to the dried blood sample or the dried blood constituent, the sample support with the sample present thereon is no longer considered to be hazardous material. From a support containing dried blood, preferably capillary blood, it is possible to detach a piece of support containing blood. This can, for example, be achieved using an apparatus as has been described in U.S. patent application Ser. No. 14/900,360 or by punching. The detached piece of support containing dried blood is commonly referred to as a “dried blood spot” or “DBS.”

A support as mentioned here can, for example, be in the form of a membrane layer or membrane which can absorb the blood or blood constituents in a liquid state and on which precisely the blood or blood constituents then dry before the support in the form of the absorptive membrane is transported.

The blood constituent absorbed by the membrane can be whole blood, more particularly capillary blood.

In order to achieve similar or comparable processing results in the course of elution or detachment of the dried blood constituent from the membrane element in the case of similar or identical patient samples so that reproducibility of the results is ensured, it is, inter alia, necessary that the membrane element, after it has been introduced into the well, does not for example float up on the surface of the liquid, meaning that its upwardly directed top side may make only insufficient contact with the liquid solution.

Int. Patent App. Pub. WO2019/089757A1 discloses a so-called multiwell plate adapter, by means of which multiple retention elements or immersion elements can be simultaneously introduced into corresponding wells of a multiwell plate. By means of such retention elements or immersion elements, relevant membrane elements can be forced to immerse into the respective liquid solution of a respective well.

### SUMMARY

It is an object of the invention to provide a simple-to-handle apparatus, by means of which membrane elements can be securely positioned in respective wells containing respective solutions in a multiwell plate, so that aspiration of the solution by means of pipetting needles can be carried out without clogging of the needle openings by the membrane elements.

According to the invention, what is therefore proposed is an apparatus for holding down respective membrane elements in respective wells of a multiwell plate. The multiwell plate has, at its top side, respective openings of the respective wells of the multiwell plate. The respective wells are each suitable for accommodation of a respective liquid solution and for accommodation of a respective membrane element.

The apparatus has furthermore, at its upper region, a support structure having respective openings of the support structure, and respective corresponding retention elements which extend downwards from the respective openings of the support structure, so that respective retention elements protrude into respective wells of the multiwell plate when the support structure has been placed onto the top side of the multiwell plate.

According to the invention, a respective retention element has multiple webs which extend downwards from the support structure and the ends of which are connected to one another at a bottom side of the apparatus by means of a connection element, wherein the connection element has a central opening. The connection element is preferably annular.

Furthermore, according to the invention, a respective retention element has multiple openings between the webs or multiple openings are formed between the webs. Said openings between the webs extend continuously from the support structure right up to the connection element. Furthermore, said openings extend continuously from the top side of the multiwell plate or from the opening of the corresponding well of the multiwell plate right up to the connection element when the support structure has been placed onto the top side of the multiwell plate. The connection element is situated preferably at the bottom side of the apparatus.

More detailed explanations will now be provided to describe one or more possible advantages of the invention.

For preferably automated processing of a biochemical method, in the course of which the patient sample must be released from the membrane element, it may be necessary, after detachment or elution of the sample from the membrane element for transfer of the sample constituents into the liquid solution, to subsequently aspirate the liquid solution from the well of the multiwell plate and to transfer it into other reaction vessels. This is done preferably by means of an aspiration needle or pipetting needle. Because the retention elements, upon immersion of the apparatus or immersion of the retention elements in the relevant wells of the multiwell plate, securely position or hold relevant membrane elements in a lower region of the well, it is possible for aspiration needles to be introduced or lowered into the respective wells of the multiwell plate and for aspiration of the liquid solution to then be performed without having to worry about clogging of the needles due to floating up of the membrane elements towards the openings of the needles. What is thus prevented by the apparatus according to the invention when transferring the eluted samples from the multiwell plate into other reaction vessels is that the membrane elements floating in the eluate do not lead to closure of the pipetting needles unnoticed. This therefore ensures correct transfer of a certain volume of the liquid by means of the pipetting needles.

The apparatus according to the invention can thus be used such that the respective retention elements are immersed into respective wells of the multiwell plate or elution plate, though care should be taken here that no liquid solution or eluate escapes over the edge of a certain well.

In the course of processing multiple patient samples or multiple membrane elements in respective wells of the multiwell plate, each well represents its own respective processing of its own respective sample. Overflow of liquid solution from one well into another well would distort the relevant processing results and give rise to false results. Because, according to the invention, respective openings are present between the webs, a respective retention element can be immersed into the liquid of a well such that the liquid solution can flow through said openings, meaning that volume displacement of the liquid solution by the retention elements is minimized and that the risk of rising of the liquid out of the well right up to the top side of the multiwell plate is therefore minimized. This minimizes the risk of overflow of liquid solution out of one well towards another well, and so cross-contamination between the respective samples of the respective wells is avoided.

Preferably since the connection element of the webs in the lower region of the apparatus has a central opening, the minimization of volume displacement of the liquid by the retention element is supported once more by said central opening, since liquid can also flow through said central opening during the introduction of the retention element into the wells or the liquids. This thus also prevents the liquid of a first sample from rising up to the top side of the multiwell plate when the retention element has been immersed into the well and prevents said liquid from possibly being transferred from one well into another well containing another liquid of another sample. Furthermore, the central opening of the connection element prevents the membrane element from floating away laterally when the retention element has been immersed, since the membrane element remains centrally positioned in the well.

Furthermore, the use of the apparatus according to the invention means that the membrane elements are always immersed into the liquid solution in a similar manner, meaning that processing results are reproducible.

Advantageous embodiments of the invention are subject matter of the dependent claims and are more particularly elucidated in the following description with some reference to the drawings.

Preferably, respective bottom sides of the connection elements form the bottom side of the apparatus. Here, the bottom side of the apparatus preferably lies in a plane or forms a plane which is parallel to a plane of the support structure.

This clearly defines and brings about a defined immersion depth of the membrane elements with respect to the plane of the support structure and thus also with respect to the top side of the multiwell plate.

Preferably, a respective retention element is designed such that a pipetting needle can be inserted downwards from above through the respectively corresponding opening at the support structure along a central axis of symmetry of the retention element right up to the preferably annular connection element. This makes it possible for the liquid solution to be transferred by means of a pipetting needle, the holding-down or immersion of the membrane element by the retention element allowing a certain immersion space or certain immersion depth for the pipetting needle.

Preferably, the webs are arranged symmetrically, preferably rotationally symmetrically, around a central axis of symmetry of the retention element. Here, the webs conically taper downwards from above or from the support structure, preferably towards the connection elements or towards the bottom side of the apparatus. This advantageously allows sufficient space between the webs and a possibly conically tapering inner side of a well, so that rising of the solution right up to the top side of the multiwell plate or right up to an opening of a well is avoided or minimized.

Preferably, the apparatus has, at respectively opposing ends of the support structure, respective mounts which can be gripped by a user. Said mounts preferably extend along a plane of the support structure. As a result, a user can advantageously grip the apparatus and then bring about the immersion of the respective retention elements into the respective wells until the support structure rests on the top side of the multiwell plate.

Preferably, each of the mounts has, at its respective bottom side, at least one spacing element, so that the spacing elements bring about spacing of a bottom side of the support structure from the top side of the multiwell plate when the apparatus or the support structure has been placed onto the top side of the multiwell plate. This advantageously avoids direct resting of the bottom side of the support structure on the top side of the multiwell plate. If this were to be the case, liquid which rises out of a well up to the bottom side of the support structure might, because of a capillary effect between the bottom side of the support structure and the top side of the multiwell plate, be transported or conducted from one well towards another well, which could cause cross-contamination of the samples. The proposed spacing elements avoid this.

Preferably, the apparatus has multiple openings arranged along a straight line and corresponding retention elements, and the apparatus has furthermore, between two openings at the bottom side of the support structure, a further spacing element which brings about spacing of the bottom side of the support structure from the top side of the multiwell plate when the support structure has been placed onto the top side of the multiwell plate. This embodiment is advantageous because, in the case of the apparatus having multiple retention elements arranged one after another, there is then spacing of the bottom side of the apparatus from the top side of the multiwell plate here as well in a central region, namely at the site of the further spacing element, such that no liquid enters a well from another well owing to capillary forces. Preferably, the further spacing element and/or the previously mentioned spacing elements bring about a distance of 2 mm between the top side of the plate or multiwell plate and the bottom side of the support structure.

Preferably, the webs of a retention element are arranged rotationally symmetrically around the central axis of symmetry of the retention element on an outer edge of the respective opening of the support structure. Here, the webs are preferably arranged at first edge sections of said edge and, furthermore, the downwardly extending openings between the webs are arranged at second edge sections of the edge. Furthermore, mutually closest, adjacent or directly adjacent edge sections of mutually adjacent openings of the support structure are preferably respectively edge sections of the second kind or second edge sections. This advantageously achieves further minimization of overflow of liquid from one well into another well, since liquid which rises up to the top side of the multiwell plate owing to capillary forces between a web and an inner side of a well cannot then find a shortest path from this one well to a next well; such a shortest path between two adjacent wells is namely the path between two nearest adjacent edge sections of the second kind.

There is proposed furthermore a kit comprising a multiwell plate and an apparatus of the kind according to the invention.

Preferably, in the case of the kit, a gap of at least 1 mm remains between a bottom side of the apparatus or bottom side of the connection element and a base side of a well when the apparatus has been inserted into the multiwell plate.

Preferably, a gap of not more than 0.9 mm, preferably 0.8 mm, particularly preferably 0.7 mm and very particularly preferably 0.6 mm remains between a web and an inner side of a well in preferably a lateral edge region of the well when the apparatus has been inserted into the multiwell plate.

This dimensioning of the gaps between the web and the inner side of the well has the advantage that the membrane element cannot move in this lateral region because it has a minimum thickness, meaning that the membrane element cannot float up, but it can nevertheless float freely below the connection element of the webs and is not clamped therebelow.

There is proposed furthermore a method for detecting an analyte in dried blood constituents, comprising the steps of providing a membrane element which comprises the dried blood constituents, introducing the membrane element into a well of a multiwell plate, introducing into the well a liquid suitable for taking up the analyte from the dried blood sample, fitting the multiwell plate together with an apparatus according to the invention so that the membrane element is positioned in a lower region of the well below the liquid surface by the apparatus, inserting a pipetting needle into the well and aspirating at least a partial volume of the liquid, transferring the partial volume of the liquid into an accommodation vessel or reaction vessel, and detecting the analyte in the partial volume of the liquid.

In the context of this application, the term “quantitative determination” is understood to mean a determination which makes it possible to state the absolute concentration of the analyte, even more preferably with a numerical value. Alternatively, the determination can be a semi-quantitative determination, in which an assignment of the concentration to a range from at least three, ideally four concentration ranges is made possible, for example negative, weakly positive and positive, or a relative determination of concentration. In particular, concentration is determined with the aid of calibrators, preference being given to two or more, preferably four units, preferably solutions or solid analytes coated on a diagnostically useful support, which each contain a known quantity of the analyte, the two or more units each comprising a different known quantity.

Quantitative determination is preferably carried out using a method selected from the group comprising immunodiffusion, immunoelectrophoresis, light scattering, agglutination and immunoassay with labelling—such as that from the group comprising immunoassay with radioactive labelling, with enzymatic labelling, more preferably ELISA, with chemiluminescence labelling, more preferably electrochemiluminescence labelling, and with immunofluorescence labelling, more preferably indirect immunofluorescence labelling—preferably with ELISA.

The dried blood constituent is eluted from the membrane element by contacting of the membrane element with the liquid which is suitable for taking up the analyte from the dried blood constituent. Possibilities here are preferably aqueous buffers having a suitable pH and salt content, for example PBS. The exact composition of the liquid and the conditions and duration of contacting can be found out by routine stabilization and optimization experiments with a view to taking up the analyte into the liquid as completely as possible and depend on the nature of the analyte. If possible, the liquid is also at the same time chosen such that it is compatible with the subsequently used method for detecting the analyte. Thereafter, the analyte of interest is detected in the liquid. What is detected is whether the analyte is present or absent or is present in a concentration above the detection limit of the detection method used. Preferably, the analyte is detected semi-quantitatively or quantitatively. Various options for carrying out the method are described in the prior art, for example Gruner, N., Stambouli, O. and Ross, R. S. (2015) Dried Blood Spots—Preparing and Processing for Use in Immunoassays and in Molecular Techniques, J. Vis. Exp 97, 52619.

In a preferred embodiment, the term “analyte”, as used herein, is understood to mean a substance which is present in the blood sample and which remains on the absorbing region of the support upon drying of the blood sample and can be transferred therefrom into another liquid for quantitative determination. Particular preference is given to substances which are highly soluble in aqueous solutions and which were dissolved in the blood sample. However, substances such as solid particles which are present in the solution in the form of a suspension are also possible. Their concentration too in the blood sample or an aqueous solution can be quantitatively determined using suitable physical measurement methods such as the detection of light scattering. In a particularly preferred embodiment, the analyte is selected from the group comprising a metabolite, a protein, a nucleic acid and a lipid and is particularly preferably an antibody, even more preferably selected from the group of classes comprising IgA, IgM, IgG and IgE, most preferably IgG. In a preferred embodiment, the antibody is an antibody from a mammal, even more preferably from a human.

In a preferred embodiment, the term “absorptive”, as used herein, is understood to mean that the material referred to in this way is capable of absorbing a blood drop, the water fraction being first absorbed and then released to the environment upon drying, with the components dissolved therein, including the analyte such as an antibody, remaining in the material and it being possible to release said components by renewed contacting with a suitable solvent, preferably an aqueous buffer. In a preferred embodiment, the absorbing region consists of non-woven polyolefin, aside from the fact that contaminants or additives customary in trade may be present.

### BRIEF DESCRIPTION OF THE DRAWINGS

In what follows, the invention will be more particularly elucidated on the basis of specific embodiments without restricting the general concept of the invention, with reference to the drawings where:

FIG. 1 shows a preferred embodiment of the apparatus according to the invention from a first perspective,

FIG. 2 shows the embodiment of the apparatus from a second perspective,

FIG. 3 shows a side view of the embodiment of the apparatus,

FIG. 4 shows a bottom view of the embodiment of the apparatus,

FIG. 5 shows a top view of the embodiment of the apparatus,

FIG. 6 shows the embodiment of the apparatus together with a multiwell plate with insertion of the apparatus into the multiwell plate,

FIG. 7 shows a sectional view of the embodiment of the apparatus together with the multiwell plate,

FIG. 8 shows a diagonal bottom view of the embodiment of the apparatus,

FIG. 9 shows a further diagonal view of the embodiment of the apparatus,

FIG. 10 shows a sectional view of a well of a multiwell plate together with a sectional view of a lower region of the embodiment of the apparatus,

FIGS. 11A-11C show retention elements of the embodiment of the apparatus that have been inserted into wells, together with various levels of a liquid solution, and

FIGS. 12 to 15 show experimental results.

### DETAILED DESCRIPTION

Various exemplary embodiments will now be described in more detail with reference to the appended drawings, in which some exemplary embodiments are depicted.

In the following description of the attached drawings, which merely show some exemplary embodiments, the same reference signs can refer to the same or comparable components. Furthermore, summarizing reference signs can be used for components and objects which occur repeatedly in an exemplary embodiment or in a drawing, but are described together with respect to one or more features. Components or objects which are described with the same or summarizing reference signs can be identical with respect to individual, multiple or all features, for example the dimensions thereof, but may also be different unless otherwise explicitly or implicitly stated by the description.

Though exemplary embodiments can be modified and altered in different ways, exemplary embodiments are depicted in the drawings as examples and are described in detail herein. However, it should be clarified that there is no intention to restrict exemplary embodiments to the forms respectively disclosed; on the contrary, exemplary embodiments are intended to cover all functional and/or structural modifications, equivalents and alternatives within the scope of the invention. Throughout the description of the drawings, the same reference signs refer to the same or similar elements.

Unless stated otherwise, all the terms used herein (including technical and scientific terms) have the same meaning ascribed thereto by an average person skilled in the art in the field to which the exemplary embodiments belong. Furthermore, it should be clarified that expressions, for example those which are defined in dictionaries in general use, are to be interpreted as if they had the meaning consistent with their meaning in the context of the relevant technology, and are not to be interpreted in an idealized or overly formal sense, unless expressly stated herein.

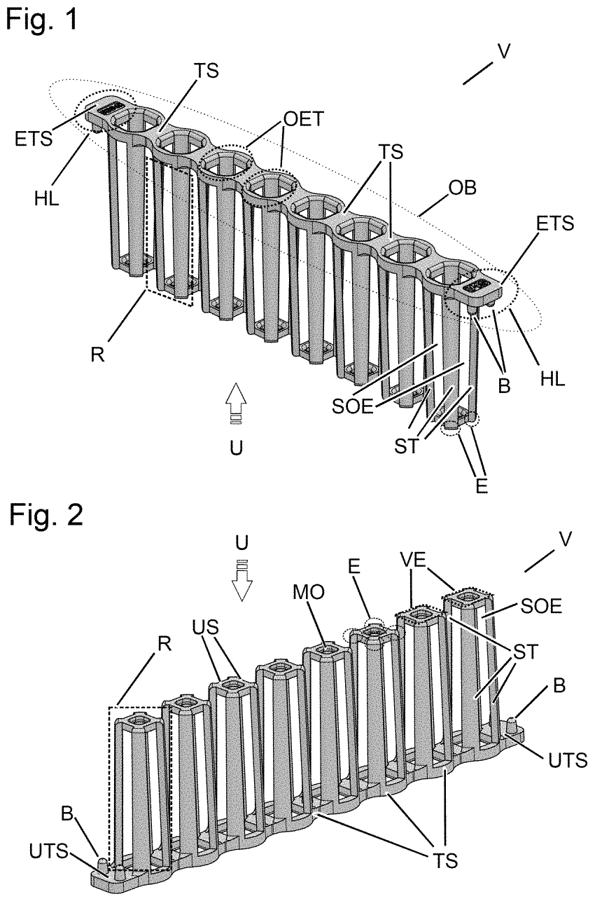

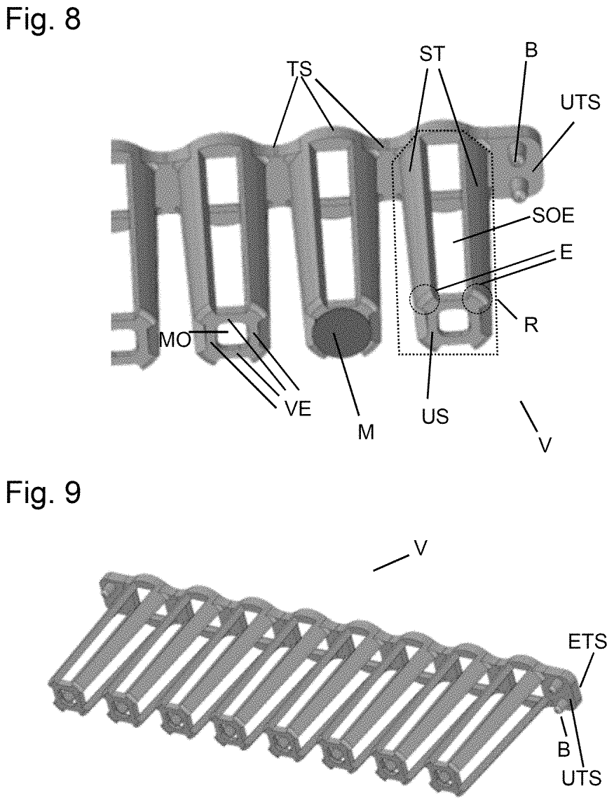

FIG. 1 shows an apparatus V for holding down membrane elements. FIG. 7 and FIG. 8 show a relevant membrane element M.

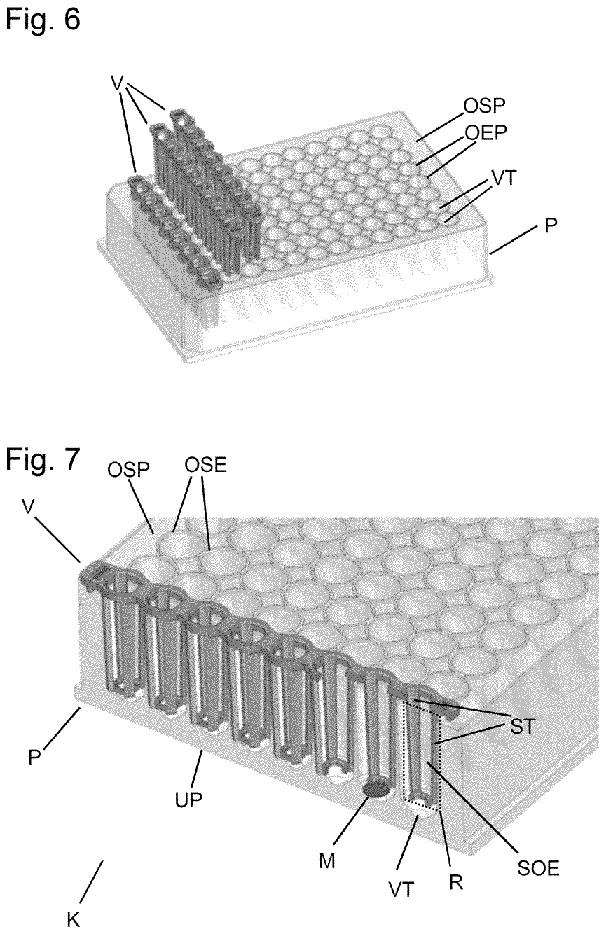

FIG. 6 shows a multiwell plate P together with the apparatus V, which has been inserted or is being inserted into the multiwell plate P.

The plate P is a multiwell plate P, preferably a microtiter plate, preferably a so-called deep-well plate. Particularly preferably, the plate P is an SBS dilution plate. The multiwell plate P is preferably composed of plastic. Preferably, the multiwell plate has a certain number of wells, said number being selected from the group consisting of six wells, twelve wells, 24 wells, 48 wells, 96 wells, 384 wells, 1536 wells and 3456 wells. Very particularly preferably, the multiwell plate is of the kind that has 96 wells.

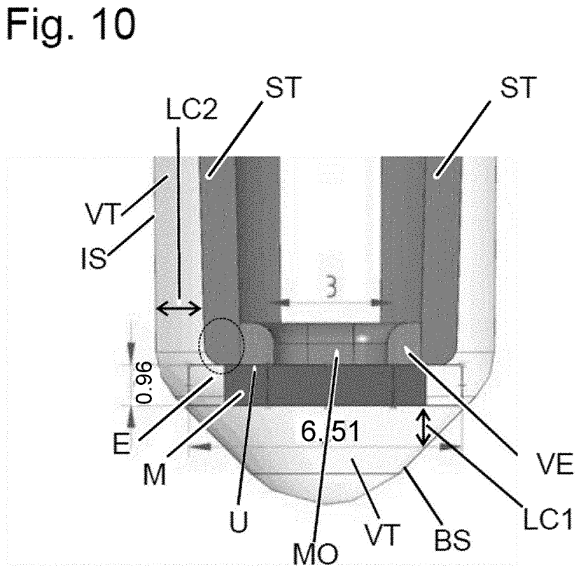

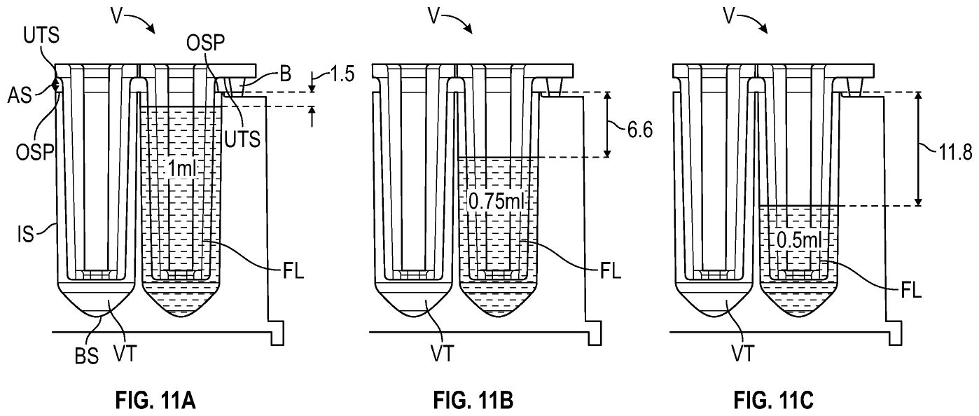

In addition to this, FIG. 7 shows once again the multiwell plate P, which has, at its top side OSP, respective openings OEP of respective wells VT. Such wells VT are also apparent in FIG. 6. Such wells VT are also depicted in FIGS. 11a, b and c, from which it is apparent that the respective wells VT are each suitable for accommodation of a liquid solution FL. The wells VT are closed or sealed at the bottom at a bottom side UP of the plate P and have a base or base side BS, as depicted in FIG. 10.

In addition to this, FIG. 10 shows an enlargement of a sub-region of a well VT together with a sectional view of a lower region of an apparatus according to the invention, it also being clear from FIG. 10 that a well VT is designed for accommodation of a membrane element M.

In FIG. 1, it is clearly depicted that the apparatus V has at its upper region OB—indicated by a dotted elliptical curve which is not part of the apparatus—a support structure TS having respective openings OET, which are each indicated by dotted circular lines. Furthermore, the apparatus V has respective retention elements R which correspond to the respective openings OET and which extend downwards.

As is apparent from FIG. 6 and FIG. 7, the apparatus V is designed such that respective retention elements R can be introduced into respective wells VT of the multiwell plate, so that the respective retention elements R protrude into the respective wells VT when the support structure TS has been placed onto the top side OSP of the multiwell plate P.

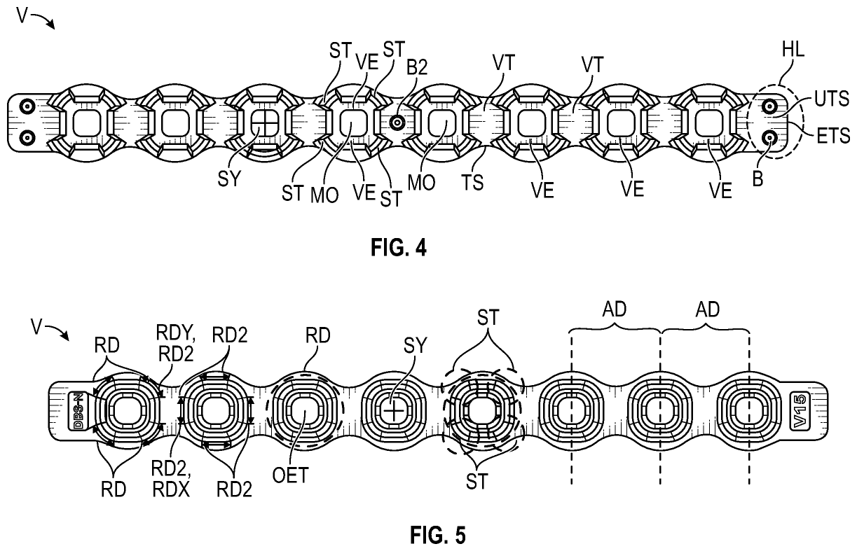

As is apparent from FIG. 1, a respective retention element R has multiple webs ST extending downwards from the support structure TS. The ends E of said webs ST are connected to one another by means of a connection element VE, which is also depicted in FIG. 10 and in FIG. 4, at a bottom side U of the apparatus V. As is likewise apparent from FIG. 4, such an preferably annular connection element VE has a central opening MO.

In addition to this, FIG. 8 as well shows, from a diagonal bottom view, a relevant connection element VE having a central opening MO.

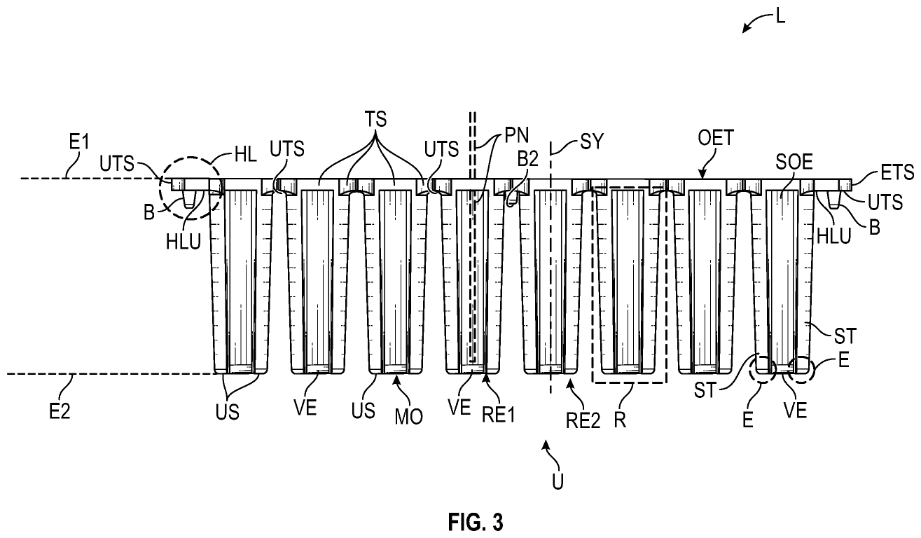

From FIG. 1, it is likewise apparent that a respective retention element R has respective openings SOE between the webs ST of the retention element R. Such an opening SOE between two webs ST has likewise been drawn in in FIG. 3 in the side view of the apparatus V. Such an opening SOE between two webs ST extends continuously from the support structure TS or the bottom side of the support structure UTS right up to the connection element VE, a bottom side US of the connection element VE forming preferably the bottom side U of the apparatus V. As is further apparent from FIG. 7, such an opening SOE between two webs ST extends from the top side OSP of the multiwell plate P right up to the connection element VE when the support structure TS has been placed onto the top side OSP of the multiwell plate P. Here, it is then preferably also the retention elements R of the apparatus V that have been inserted into the wells VT.

The presently implemented dimensioning of the openings SOE between two webs ST of a retention element R results in maximization of through-flow ability of liquid solution FL—see FIGS. 11A-11C—when the retention elements R have been introduced into the wells VT, as depicted in FIG. 7. This minimizes rising of liquid solution right up to the top side OSP of the multiwell plate P.

FIG. 2 shows, for a retention element R, the bottom side US of a connection element VE. Such a bottom side US of a connection element VE is drawn in again in FIG. 3. The respective bottom sides US of the connection elements VE form the bottom side U of the apparatus V. The bottom side U of the apparatus V lies in a plane E2 or forms a plane E2 which is parallel to a plane E1 of the support structure TS.

If the retention elements R are introduced into relevant wells VT of the multiwell plate P, then what is effected in a respective well VT is that a membrane element M present in the well VT is held or positioned in a lower region of the well VT by means of the bottom side US of a connection element VE or the bottom side U of the apparatus V. This is preferably effected for multiple membrane elements M in an individual well VT.

In relation to this, FIG. 10 shows a sectional view of a well VT of a multiwell plate in the case of a retention element R present in the well VT. Here too, it is apparent that the webs ST are held together at their ends E by the connection element VE, as indicated by a dotted elliptical curve. The bottom side U of the apparatus V positions the membrane element M within the well VT.

Positioning of the membrane element M in the manner depicted in FIG. 10 makes it possible for a pipetting needle to be inserted or introduced from above into the opening OET of the support structure and the opening OEP of the well VT up to a preferred depth which is exactly somewhat smaller than the depth of the connection element VE, and so clogging of an opening of the pipetting needle by the membrane element M is also then prevented.

The membrane element M can still move in the lower region of the well VT and its top side is also sufficiently contacted or soaked by liquid.

Furthermore, volume displacement of the liquid FL by the apparatus V is minimized preferably by the continuous extension of the openings SOE between the webs ST and by the central opening MO of the connection element VE. If one or more openings were to be present merely in a lower region of a retention element R in order to allow flow of a liquid through the apparatus, then immersion of the retention elements into the wells would possibly bring about an excessively large volume displacement of the liquid FL, which could cause rising of the liquid FL right up to the top side OSP of the multiwell plate OE and thus cross-contamination between the various samples of various wells.

FIG. 3 shows an example in which a pipetting needle PN has been inserted downwards from above through the opening OET of the support structure TS along an axis of symmetry SY of the retention element R right up to the connection element VE.

As is apparent from FIG. 11A, the wells VT preferably have a conical taper in a downward direction towards the base side BS from the top side OSP of the plate P.

As is apparent from FIG. 1 and FIG. 5, the webs ST of a retention element R are attached to an edge region RD of an opening OET of the support structure TS and extend downwards from said edge region RD of an opening OET. The entire opening region OET is thereby provided for introduction of the pipetting needle PN from FIG. 3, and so what is achieved is a relatively high tolerance regarding variance of the needle position or positioning of the needle PN in an xy plane in which the support structure runs or along which the support structure runs. Slight alignment variances in the position of the pipetting needle PN in this xy plane or the plane of the support structure or the plane of the opening OET are tolerable as a result.

As is further apparent from FIG. 5, the webs ST are arranged symmetrically, preferably rotationally symmetrically, around a central axis of symmetry SY of the retention element R. The axis of symmetry SY is preferably perpendicular to the plane E1 of the support structure TS. As is apparent from FIG. 3 and FIG. 7, the webs ST conically taper downwards towards the bottom side U of the apparatus V from above from the support structure TS. FIG. 11A illustrates that, in the case of conically tapering wells VT, sufficient space between the webs ST and the inner side IS of a well is thereby provided. What is thereby minimized in the case of conically tapering wells, owing to the conical tapering of the webs, is a capillary effect between the inner sides IS of the well VT and the webs ST, and so rising of the solution right up to the openings OEP of the multiwell plate P due to capillary forces is minimized.

FIG. 1 shows, as does FIG. 4, that the apparatus V has, at respectively opposing ends ETS of the support structure TS, respective mounts HL at which the user can grip the apparatus V. As a result, a user can introduce the retention elements R into the respective wells VT of the plate P in a particularly simple manner as depicted in FIG. 6 and, in the case of provision of multiple retention elements R in an individual apparatus V, the simultaneous introduction of the retention elements R into the wells VT can thus be made possible. Such mounts HL are also depicted in detail in FIG. 3 and identified in FIG. 2.

Each of the mounts HL has at its respective bottom side HLU—see FIG. 3 and FIG. 4—at least one spacing element B, so that the spacing elements B bring about spacing of a bottom side UTS of the support structure TS from the top side OSP of the multiwell plate P when the support structure TS has been placed onto the top side OSP of the multiwell plate P. This can also be very easily identified in FIGS. 11A-11C, since it is apparent there that a spacing element B brings about a distance AS between the bottom side UTS of the support structure and the top side OSP of the plate. Such a distance is preferably at least 2 mm in order to minimize transport of liquid from one well into another well owing to capillary forces owing to a capillary effect between the bottom side UTS of the support structure and the top side OSP of the plate.

FIGS. 1 and 5 show together that the apparatus V preferably has multiple openings OET arranged along a straight line and corresponding retention elements R. Preferably, said retention elements R are arranged relative to one another at regular, equidistant distances AD.

In FIG. 3, two retention elements RE1, RE2 are identified in a central region of the apparatus V. The apparatus V has furthermore, between the retention elements RE1, RE2 at the bottom side UTS of the support structure, as also shown in FIG. 3, a spacing element B2 which brings about spacing of the bottom side UTS of the support structure TS from the top side OSP of the multiwell plate P when the support structure TS has been placed onto the top side OSP of the multiwell plate P.

As a result, spacing between the bottom side UTS of the support structure TS and top side OSP of the plate P is also assisted in a further, central region of the apparatus V, since, in the case of the presence of multiple retention elements R in a straight line, bendability of the apparatus V might otherwise lead to reduction of a distance between the bottom side UTS of the support structure TS and the top side OSP of the plate P. This too minimizes overflow of liquid from one well into another well owing to capillary forces between the bottom side UTS of the support structure TS and the top side OSP of the plate P.

From FIG. 5, it is apparent that a respective opening OET has an outer edge RD. The webs ST of a retention element are arranged rotationally symmetrically around the central axis of symmetry SY of the retention element R along the outer edge RD of a respective opening OET.

Drawn in in a left-hand region of FIG. 5 are first edge sections RD1, at which the webs ST are arranged. Furthermore, there are second edge sections RD2, at which the downwardly extending openings SOE between the webs ST are situated or at which they are arranged.

From FIG. 5, it is thus apparent that mutually closest, adjacent edge sections RDX, RDY of adjacent openings of the support structure TS are respectively second edge sections RD2. They are thus edge sections RD2 having downwardly extending openings SOE between the webs ST. This further minimizes overflow of liquid from a first well into another well, since liquid which rises up to the top side OSP of the multiwell plate owing to capillary forces between a web ST and an inner side IS of a well VT cannot then find the shortest path towards the other well. The shortest path is provided between two edge sections RD2 of the second kind, also entered as exemplary edge sections RDX, RDY, at which it is precisely the openings SOE in particular that are arranged. The webs ST are not arranged at said edge sections RDX, RDY.

There is further proposed a kit K, as entered in FIG. 6 and FIG. 7, which comprises a multiwell plate P and a proposed apparatus V.

FIG. 10 shows that a gap LC1 of preferably at least 1 mm remains between a bottom side U of the apparatus and a base side BS of a well VT when the apparatus V has been inserted into the multiwell plate P. As a result, the membrane element M has sufficient freedom of movement available during the elution or detachment of the dried blood constituents.

As further depicted in FIG. 10, a gap LC2 of not more than 0.9 mm, preferably 0.8 mm, otherwise preferably 0.7 mm and very particularly preferably 0.6 mm preferably remains between a web ST and an inner side IS of a well VT when the apparatus V has been inserted into the multiwell plate P.

Since a membrane element M has a thickness of preferably 0.96 mm for example, choosing the presently proposed dimensions of the gap LC2 can prevent rising of the membrane element M between web ST and inner side IS of the well VT owing to buoyancy.

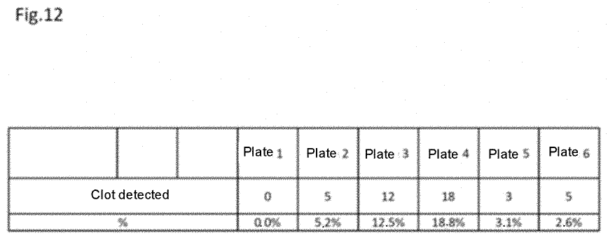

FIG. 12 shows results relating to aspiration of liquids from wells of multiwell plates for the cases of either no membrane element being present in a well (Plate 1) or else a membrane element being respectively present in a respective well (Plate 2 to Plate 6). All these aspiration tests were carried out without the proposed apparatus for holding down membrane elements. All the plates used here, Plate 1 to Plate 6, each had 96 wells and were of the type “96-Well Nunc Plates, F-Bottom, uncoated.”

In the case of each plate, 96 aspirations were carried out for respective wells. In the case of Plate 1, needle clogging or a clot event was detected for 0 aspiration operations. In the case of Plate 2, needle clogging or a clot event was detected for 5 aspiration operations from a total of 96 aspiration operations. In the case of Plate 3, this was the case for 12 aspiration operations. In the case of Plate 4, this was the case for 18 aspiration operations. In the case of Plate 5, this was the case for 3 aspiration operations. In the case of Plate 6, this was the case for 5 aspiration operations. The results from FIG. 12 clearly show the need to hold down membrane elements in order to avoid clogging of an aspiration needle or to avoid clot events. This avoidance of clogging of an aspiration needle is especially advantageous because aspiration of an incorrect liquid volume can lead to incorrect processing results when detecting the analyte.

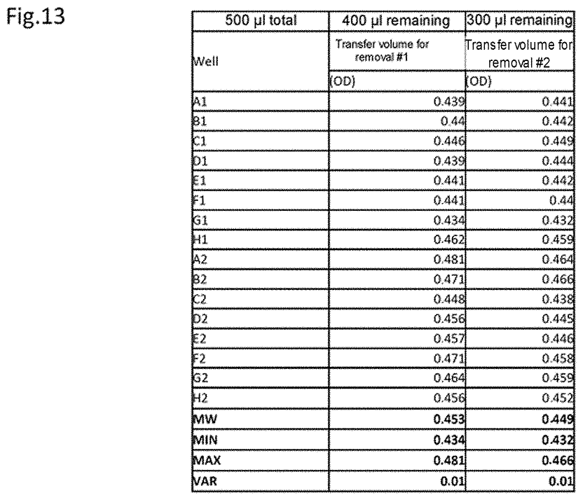

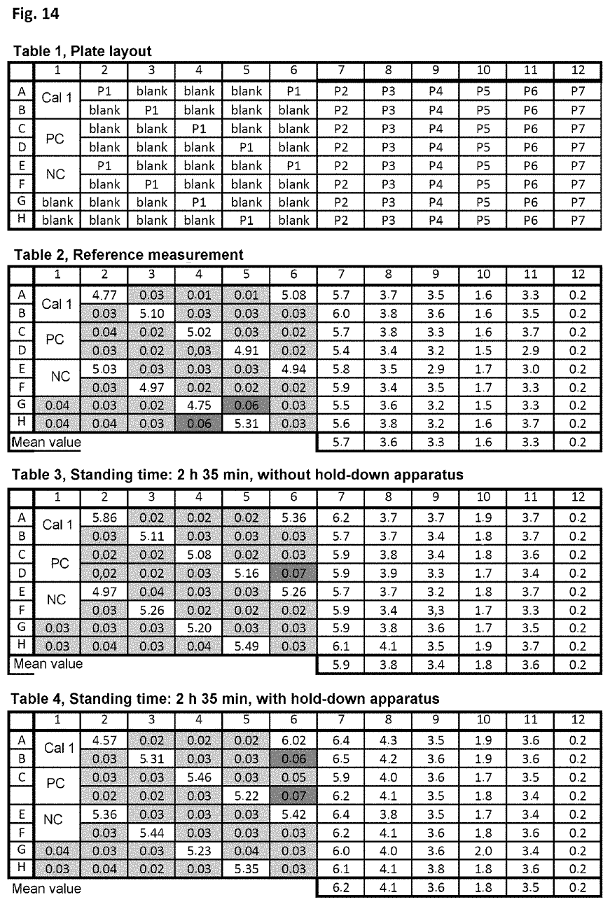

FIG. 13 shows experimental results in the case of aspiration from 16 different wells labelled A1 to H2 of a multiwell plate of the type “Riplate medio 1 ml” from Ritter. In all these wells A1 to H2, two membrane elements and 500 ml of liquid in the form of “Buffer Blue” were present in each case. The multiwell plate was first incubated in an incubator and then subsequently shaken on a rotating shaker. A proposed apparatus for holding down or retaining membrane elements was then inserted into the wells of the multiwell plate. Aspiration from the respective wells A1 to H2 was then carried out by means of the product “EUROLabWorkstation ELISA” from EUROIMMUN Medizinische Labordiagnostika AG. In a first removal step, a quantity of 100 μl was initially aspirated from each well, leaving a remainder of 400 μl. In a second removal step, a quantity of 100 μl was then aspirated again, leaving a remaining volume of 300 μl. Each of the aspirated volumes was then transferred into a separate container and the optical density (OD) of the transferred volume was measured proportionally.

For a respective well A1 to H2, the respectively measured optical densities are listed in the middle column for the first removal step and in the right-hand column for the second removal step. Optical density is a measure or indicator of the aspirated and transferred sample quantity or the aspirated and transferred volume. The results show that the measured optical densities have very similar values across all volumes for the two steps and yield a variance of merely 0.01. The very low variance of this optical density indicates that almost identical sample volumes were also aspirated and transferred in the respective removal steps 1 and 2 for all wells A1 to H2. This shows that significant clogging of the aspiration needle by a membrane element did not occur in any case.

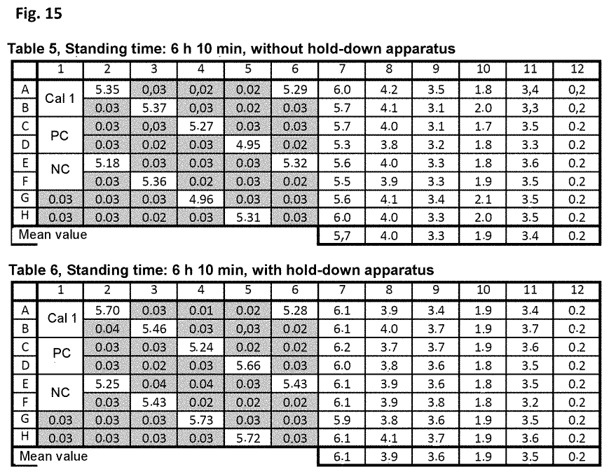

FIGS. 14 and 15 show, in Table 1 to Table 6, further results regarding possible overflow of sample liquid from one well into another well. Table 1 shows a layout of a multiwell plate with rows A to H and columns 1 to 12, as is customary for a 96-well multiwell plate. Wells A1 and B1 were each filled with calibrator liquid. Two further wells C1, D1 were filled with positive controls. Two further wells E1, F1 were filled with negative controls. Wells G1 and H1 were each only filled with so-called buffer liquid. Further different wells were filled with samples having the index P1 to P7. Wells specified “blank” were only filled with buffer liquid in each case.

A reference measurement of optical density was then carried out for each of the respective wells, and what is reported by the measurement values shown in Table 2 is relative optical density as a quotient of the optical density of the respective sample of the respective well divided by the optical density of the calibration liquid from wells A1 and B1.

It is apparent from Table 2 that wells containing actual samples having index P1 to P7 each have, directly after filling, relative optical density values which are distinctly higher than those of those wells which only comprise buffer liquid.

Here, measurement values with light grey marking have an optical density quotient of less than 0.05. Here, measurement values with dark grey marking have an optical density quotient of less than 0.2.

Table 3 from FIG. 14 shows measured quotients of relative optical densities for the respective wells after a standing time of 2 hours and 35 minutes without insertion of the proposed apparatus for holding down or retaining membrane elements. It was thus not possible to bring about carry-over of liquid from one well into another by such an apparatus. Table 4 shows relevant relative optical densities for the respective wells in the case of insertion of the proposed apparatus into the respective wells for a standing time of 2 hours and 35 minutes. A comparison of the measurement results from Table 3 and Table 4 clearly shows that the proposed apparatus did not bring about significant carry-over of sample liquid from one well into another. Furthermore, relative optical density values for wells which only comprised sample liquid did not rise noticeably in any of the cases, with or without use of the proposed apparatus.

FIG. 15 shows relevant results for a standing time of 6 hours and 10 minutes without use of the proposed apparatus in Table 5 and with use of the proposed apparatus in Table 6. Significant carry-over of liquid from one well into another cannot be observed here either. This underlines the usability of the proposed apparatus.

All references, including patents, patent applications and publications cited herein are incorporated herein by reference in their entirety and for all purposes to the same extent as if each individual publication or patent or patent application was specifically and individually indicated to be incorporated by reference in its entirety for all purposes.

What is claimed is:

1. An apparatus for holding down respective membrane elements in respective wells of a multiwell plate, the multiwell plate having, at its top side, respective openings of the respective wells, the respective wells each suitable for accommodation of a respective liquid solution and for accommodation of the respective membrane elements, the apparatus comprising:
an upper region comprising a support structure having respective openings and respective corresponding retention elements which extend downwards from the respective openings of the support structure, so that respective retention elements protrude into respective wells when the support structure has been placed onto the top side of the multiwell plate,
wherein a respective retention element:
comprises multiple webs which extend downwards from the support structure and the ends of which are connected to one another at a bottom side of the apparatus by means of an annular connection element which has a central opening, and
comprises multiple openings between the webs, which openings extend continuously from the support structure right up to the connection element and which openings furthermore extend continuously from the top side of the multiwell plate right up to the connection element when the support structure has been placed onto the top side of the multiwell plate.

2. The apparatus according to claim 1, wherein:
respective bottom sides of the connection elements form a bottom side of the apparatus, and
the bottom side of the apparatus lies in a plane or forms a plane which is parallel to a plane of the support structure.

3. The apparatus according to claim 1, wherein a respective retention element is designed such that a pipetting needle can be inserted downwards from above through the respectively corresponding opening at the support structure along a central axis of symmetry of the retention element right up to the annular connection element.

4. The apparatus according to claim 1, wherein:
the webs are arranged symmetrically around a central axis of symmetry of the retention element, and
the webs conically taper downwards from above.

5. The apparatus according to claim 4, wherein:
the webs of a retention element are arranged rotationally symmetrically around the central axis of symmetry of the retention element on an outer edge of the respective opening of the support structure, and
the webs are arranged at first edge sections of the edge and wherein the downwardly extending openings between the webs are arranged at second edge sections of the edge, such that mutually closest, adjacent edge sections of adjacent openings of the support structure are respectively second edge sections.

6. The apparatus according to claim 1, wherein the apparatus has, at respectively opposing ends of the support structure, respective mounts which can be gripped by a user.

7. The apparatus according to claim 6, wherein each of the mounts has, at its respective bottom side, at least one spacing element, so that the spacing elements bring about spacing of a bottom side of the support structure from the top side of the multiwell plate when the apparatus has been placed onto the top side of the multiwell plate.

8. The apparatus according to claim 7, wherein:
the apparatus has multiple openings arranged along a straight line and corresponding retention elements, and
the apparatus has furthermore, between two openings at the bottom side of the support structure, a further spacing element which brings about spacing of the bottom side of the support structure from the top side of the multiwell plate when the apparatus has been placed onto the top side of the multiwell plate.

9. A kit comprising:
a multiwell plate having, at its top side, respective openings of the respective wells, the respective wells each suitable for accommodation of a respective liquid solution and for accommodation of respective membrane elements; and
an apparatus for holding down the respective membrane elements in the respective wells of the multiwell plate, the apparatus comprising:
an upper region comprising a support structure having respective openings and respective corresponding retention elements which extend downwards from the respective openings of the support structure, so that respective retention elements protrude into respective wells when the support structure has been placed onto the top side of the multiwell plate,
wherein a respective retention element comprises:
multiple webs which extend downwards from the support structure and the ends of which are connected to one another at a bottom side of the apparatus by means of an annular connection element which has a central opening; and
multiple openings between the webs, which openings extend continuously from the support structure right up to the connection element and which openings furthermore extend continuously from the top side of the multiwell plate right up to the connection element when the support structure has been placed onto the top side of the multiwell plate.

10. The kit according to claim 9, wherein a gap of at least 1 mm remains between a bottom side of the apparatus and a base side of a well when the apparatus has been inserted into the multiwell plate.

11. The kit according to claim 9, wherein a gap of not more than 0.9 mm remains between a web and an inner side of a well when the apparatus has been inserted into the multiwell plate.

12. The kit according to claim 9, wherein:
respective bottom sides of the connection elements form a bottom side of the apparatus, and
the bottom side of the apparatus lies in a plane or forms a plane which is parallel to a plane of the support structure.

13. The kit according to claim 9, wherein a respective retention element is designed such that a pipetting needle can be inserted downwards from above through the respectively corresponding opening at the support structure along a central axis of symmetry of the retention element right up to the annular connection element.

14. The kit according to claim 9, wherein:
the webs are arranged symmetrically around a central axis of symmetry of the retention element, and
the webs conically taper downwards from above.

15. The kit according to claim 14,
wherein the webs of a retention element are arranged rotationally symmetrically around the central axis of symmetry of the retention element on an outer edge of the respective opening of the support structure, and
wherein the webs are arranged at first edge sections of the edge and wherein the downwardly extending openings between the webs are arranged at second edge sections of the edge, such that mutually closest, adjacent edge sections of adjacent openings of the support structure are respectively second edge sections.

16. The kit according to claim 9, wherein the apparatus has, at respectively opposing ends of the support structure, respective mounts which can be gripped by a user.

17. The kit according to claim 16, wherein each of the mounts has, at its respective bottom side, at least one spacing element, so that the spacing elements bring about spacing of a bottom side of the support structure from the top side of the multiwell plate when the apparatus has been placed onto the top side of the multiwell plate.

18. The kit according to claim 17, wherein:
the apparatus has multiple openings arranged along a straight line and corresponding retention elements, and
the apparatus has furthermore, between two openings at the bottom side of the support structure, a further spacing element which brings about spacing of the bottom side of the support structure from the top side of the multiwell plate when the apparatus has been placed onto the top side of the multiwell plate.

19. A method for detecting an analyte in dried blood constituents, comprising the steps of:
providing a membrane element which comprises the dried blood constituents;
introducing the membrane element into a well of a multiwell plate;
introducing into the well a liquid suitable for taking up the analyte from the dried blood sample;
fitting the multiwell plate together with an apparatus so that the membrane element is positioned in a lower region of the well below the liquid surface by the apparatus;
inserting a pipetting needle into the well and aspirating at least a partial volume of the liquid;
transferring the partial volume of the liquid into an accommodation vessel; and
detecting the analyte in the partial volume of the liquid.
